(19) United States
(12) Patent Application Publication
(10) Pub. No.: US 2019/0247007 A1
(43) Pub. Date: Aug. 15, 2019

(54) PROTECTIVE CASE

(71) Applicant: Vitalchains Corporation, Menlo Park, CA (US)

(72) Inventors: Kun-Hsi TSAI, Zhubei City (TW); Tzu-Chih LIN, Zhubei City (TW)

(21) Appl. No.: 16/259,678

(22) Filed: Jan. 28, 2019

Related U.S. Application Data
(60) Provisional application No. 62/629,131, filed on Feb. 12, 2018.

(51) Int. Cl. A61B 7/02; A61B 50/30

(52) U.S. Cl. CPC A61B 7/02; A61B 50/30

(57) ABSTRACT

A protective case includes a first housing and a second housing. The first housing includes a first cavity portion and a first engaging portion. The first cavity portion has a first opening. The first engaging portion is connected to the first opening. The second housing includes a second cavity portion, a second engaging portion, and a bump. The second cavity portion has a second opening. The second engaging portion is connected to the second opening. The second engaging portion is detachably engaged with the first engaging portion, such that the first cavity portion and the second cavity portion form an accommodating space therebetween. The bump is connected to the second cavity portion and protrudes toward the second opening.

### CROSS-REFERENCE TO RELATED APPLICATION

This application claims priority to U.S. Provisional Application Ser. No. 62/629,131, filed Feb. 12, 2018, which is herein incorporated by reference in its entirety.

### BACKGROUND

### Field of Invention

The present invention relates to a protective case. More particularly, the present invention relates to the protective case for a stethoscope.

### Description of Related Art

Currently, a stethoscope is packaged in a retail box to be carried to a variety of exhibitions. However, the inconvenience of shipping is resulted due to the occupied volume of the retail box. Therefore, salespeople often pack stethoscopes with cloth instead of retail boxes. Without the protection of the retail boxes, the chest pieces of the stethoscope are easily got damaged during shipment, which results in expensive repair costs.

Accordingly, how to provide a protective case to solve the aforementioned problems becomes an important issue to be solved by those in the industry.

### SUMMARY

The invention provides a protective case for protecting a stethoscope and easy to be carried.

According to an embodiment of the disclosure, the protective case includes a first housing and a second housing. The first housing includes a first cavity portion and a first engaging portion. The first cavity portion has a first opening. The first engaging portion is connected to the first opening. The second housing includes a second cavity portion, a second engaging portion, and a bump. The second cavity portion has a second opening. The second engaging portion is connected to the second opening. The second engaging portion is detachably engaged with the first engaging portion, such that the first cavity portion and the second cavity portion form an accommodating space therebetween. The bump is connected to the second cavity portion and protrudes toward the second opening.

In an embodiment of the disclosure, one of the first engaging portion and the second engaging portion has a protrusion. Another of the first engaging portion and the second engaging portion has a slot. The slot is configured to be detachably engaged with the protrusion.

In an embodiment of the disclosure, the first housing further includes a first connecting portion. The first connecting portion is connected to the first engaging portion. The second housing further includes a second connecting portion. The second connecting portion is connected to the second engaging portion and the first connecting portion. The first connecting portion and the second connecting portion form a junction therebetween.

In an embodiment of the disclosure, the bump includes a third cavity portion. The third cavity portion has a third opening. The second opening and the third opening are respectively located at opposite sides of the second housing.

In an embodiment of the disclosure, the first housing further includes a notch. The notch is located at an edge of the first opening. The notch extends from an inner wall of the first cavity portion to an outer edge of the first engaging portion.

In an embodiment of the disclosure, the second housing further includes a notch located at an edge of the second opening. The notch extends from an inner wall of the second cavity portion to an outer edge of the second engaging portion.

In an embodiment of the disclosure, each of the first cavity portion and the second cavity portion has an annular edge.

In an embodiment of the disclosure, the first housing further includes a first buckle portion located at an edge of the first engaging portion. The second housing further includes a second buckle portion located at an edge of the second engaging portion. The first buckle portion is detachably buckled with the second buckle portion.

Accordingly, in the protective case of the present disclosure, the protective case is a package structure that is light and easy to carry. Therefore, using the protective piece of the present disclosure to pack the stethoscope is convenient for a user to take the stethoscope to everywhere without crashing the chest piece of the stethoscope. The bump located in the protective case helps to position the chest piece, so as to prevent the chest piece from colliding with the protective piece and thereby preventing from causing damage to the chest piece.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIG. 1 is a perspective view of a protective case according to some embodiments of the disclosure, in which the protective case is in an expanded state;

FIG. 2 is a perspective view of the protective case according to some embodiments of the disclosure, in which the protective case is in a collapsed state;

FIG. 3 is a cross-section view of the protective case taken along line 3-3 in FIG. 2;

FIG. 4 is a cross-section view of the protective case taken along line 4-4 in FIG. 2, in which a stethoscope with a single head is accommodated in the protective case; and

FIG. 5 is a cross-section view of the protective case taken along line 4-4 in FIG. 2, in which a stethoscope with a double head is accommodated in the protective case.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

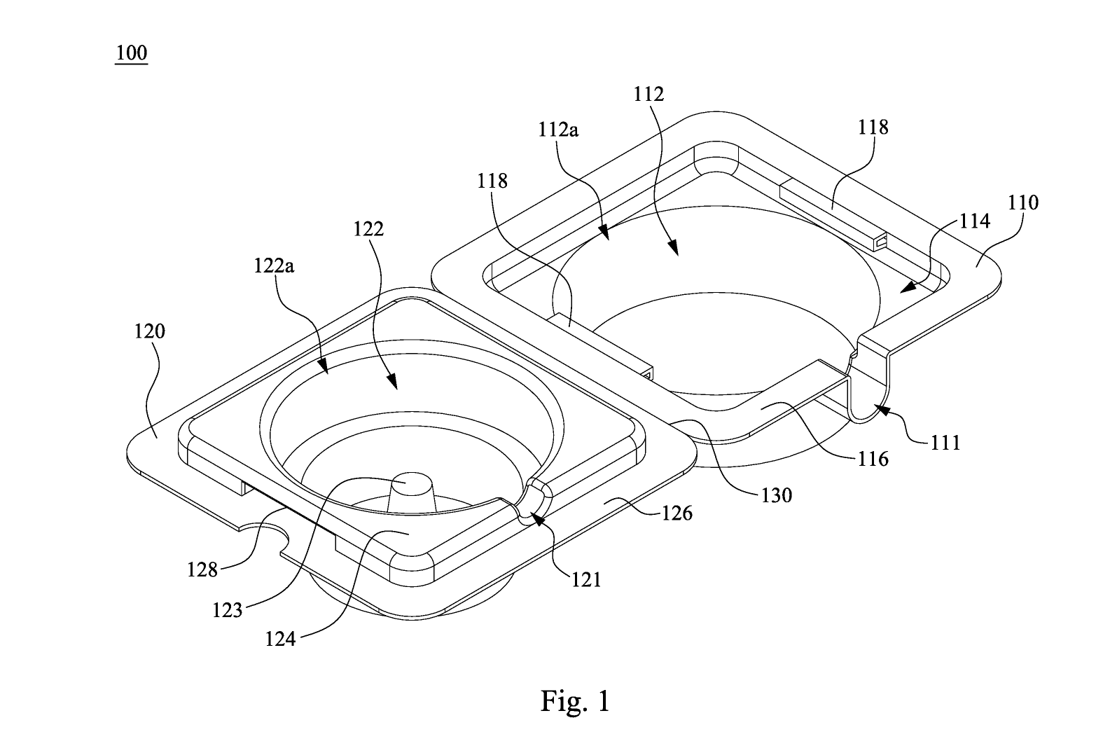

Reference is made to FIG. 1. FIG. 1 is a perspective view of a protective case 100 according to some embodiments of the disclosure, in which the protective case is 100 in an expanded state.

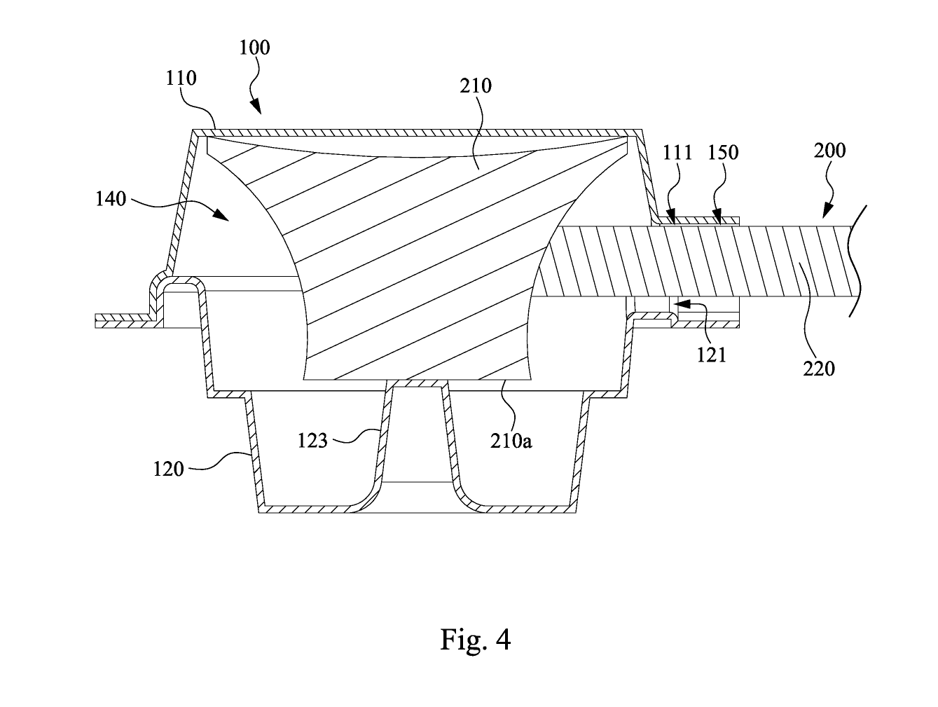

As shown in FIG. 1, the protective case 100 is a case housing used to accommodate a chest piece 210 of a stethoscope 200 (as shown in FIG. 4). The protective case 100 includes a first housing 110 and a second housing 120. The first housing 110 includes a first cavity portion 112, a first engaging portion 114, a first connecting portion 116, two first buckle portions 118, and a first notch 111. The first cavity portion 112 has a first opening 112a. The first engaging portion 114 is connected to the first opening 112a. The first engaging portion 114 is a slot. The first connecting portion 116 is connected to and surrounds an outer edge of the first engaging portion 114. The two first buckle portions 118 are respectively located at two opposite inner walls of the first engaging portion 114 and protrude toward the first opening 112a of the first cavity portion 112. The first notch 111 is located at an edge of the first opening 112a and extends from an inner wall of the first cavity portion 112 to an outer edge of the first connecting portion 116 through the first engaging portion 114.

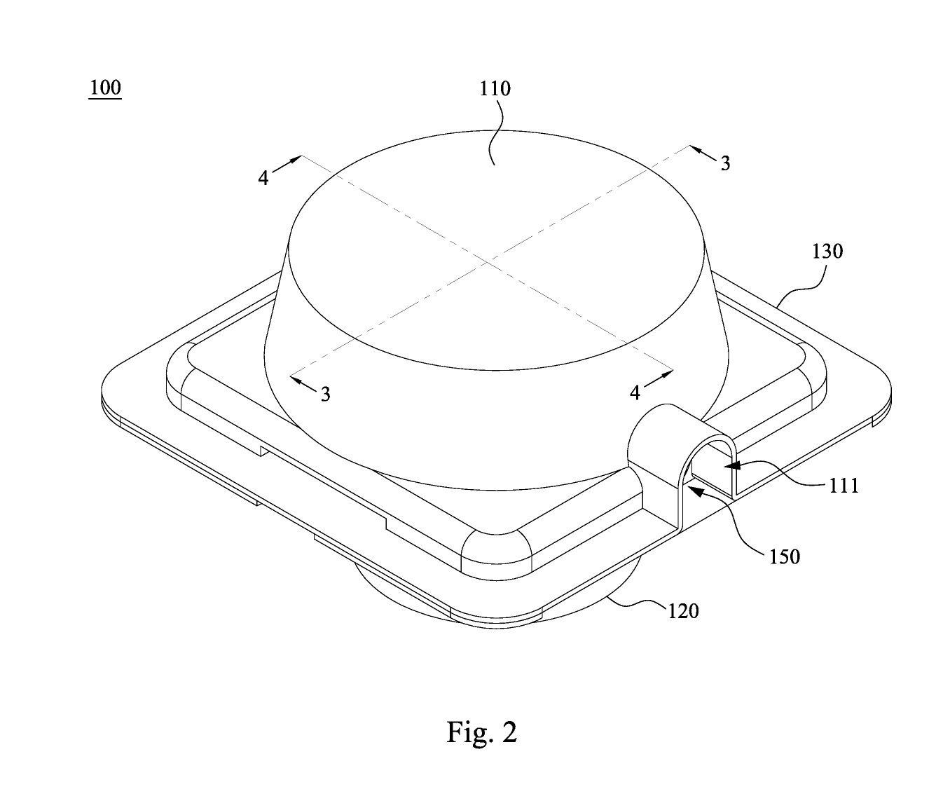

The second housing 120 includes a second cavity portion 122, a second engaging portion 124, a second connecting portion 126, two second buckle portions 128, a second notch 121, and a bump 123. The second cavity portion 122 has a second opening 122a. The second engaging portion 124 is connected to the second opening 122a. The second engaging portion 124 is a protrusion. The first engaging portion 114 which is the slot is configured to be detachably engaged with the second engaging portion 124 which is the protrusion. The second connecting portion 126 is connected to and surrounds an outer edge of the second engaging portion 124. A side of the second connecting portion 126 is further connected to a side of the first connecting portion 116. Thereby, a junction 130 is formed between the side of the first connecting portion 116 and the side of the second connecting portion 126. The junction 130 is bendable such that the protective case 100 can be selectively deformed to an expanded state (as shown in FIG. 1) or a collapsed state (as shown in FIG. 2). The two second buckle portions 128 are respectively located at two opposite outer walls of the second engaging portion 124 corresponding to positions of the first buckle portions 118. The two second buckle portions 128 are recesses recessing toward the second opening 122a. The second buckle portions 128 are configured to be detachably engaged with the first buckle portions 118 respectively. The second notch 121 is located at an edge of the second opening 122a and extends from an inner wall of the second cavity portion 122 to the outer edge of the second engaging portion 124. The bump 123 is connected to a bottom surface of the second cavity portion 122 and protrudes toward the second opening 122a. Both of the first opening 112a and the second opening 122a have annular edges. Specifically, the annular edges of the first opening 112a and the second opening 122a are circular edges which is fit with a circular edge of a chest piece 210 of a stethoscope 200, but the disclosure should not be limited in this regard.

In some other embodiments, the first engaging portion 114 is a protrusion and the second engaging portion 124 is a slot. In the embodiment, the first buckle portions 118 are recesses recessing from two outer walls of the first engaging portion 114. The second buckle portions 128 are respectively located at two inner walls of the second engaging portion 124 corresponding to the positions of the first buckle portion 118. The second buckle portions 128 protrude toward the second opening 122a. In some embodiments, a lateral width of the slot is larger than a lateral width of the protrusion. In some embodiments, a depth of the slot is larger than a height of the protrusion.

In some embodiments, numbers and positions of the first and second buckle portions 118, 128 should not be limited by the embodiments illustrated by FIG. 1 and can be adjusted according to actual needs.

In some embodiments, the first and second connecting portions 116, 126 continuously surround the first and second engaging portions 114, 124 respectively, but the disclosure is not limited in this regard. In some other embodiments, the first and second connecting portions 116, 126 do not continuously surround the first and second engaging portions 114, 124 respectively. For example, the first and second connecting portions 116, 126 may extend partially along the sides of the first and second engaging portions 114, 124 respectively.

In some other embodiments, the junction 130 is formed between a corner of the first connecting portion 116 and a corner of the second connecting portion 126, but the disclosure should not be limited in this regard.

In some other embodiments, the first housing 110 and the second housing 120 are separate. That is, the first connecting portion 116 of the first housing 110 and the second connecting portion 126 of the second housing 120 are not connected to each other. In such embodiments, the first housing 110 and the second housing 120 may be fixed to each other by engaging the first and second engaging portions 114, 124 to each other, but the disclosure should not be limited in this regard.

In some embodiments, the first and second housings 110, 120 are formed by injection molding or blister molding for example, but the disclosure should not be limited in this regard.

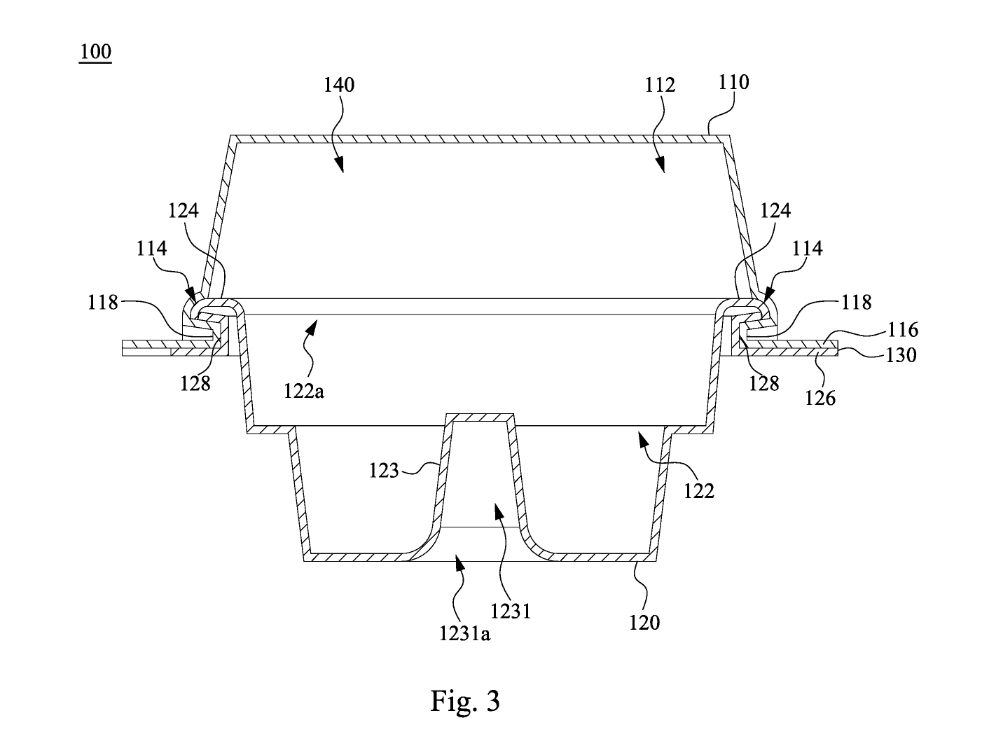

Reference is made to FIGS. 2 and 3. FIG. 2 is a perspective view of the protective case 100 according to some embodiments of the disclosure, in which the protective case 100 is in a collapsed state. FIG. 3 is a cross-section view of the protective case 100 taken along line 3-3 in FIG. 2.

As shown in FIGS. 2 and 3, when the protective case 100 is in the collapsed state, the first engaging portion 114 is engaged with the second engaging portion 124. Specifically, the second engaging portion 124 which is the protrusion is fitted in the first engaging portion 114 which is the slot. The first buckle portions 118 are buckled with the second buckle portions 128 respectively. As such, a surface of the first connecting portion 116 is in contact with a surface of the second connecting portion 126. The first cavity portion 112 is connected to the second cavity portion 122. The first notch 111 is connected to the second notch 121. A first accommodating space 140 is formed between the first cavity portion 112 and the second cavity portion 122. A second accommodating space 150 is formed between the first notch 111 and the second notch 121 (the second notch 121 is omitted in FIG. 2 owing to the angle of view). The first accommodating space 140 is configured to accommodate the chest piece 210 of the stethoscope 200. The second accommodating space 150 is configured to accommodate a tube 220 (referring to FIG. 4) of the stethoscope 200. The first and second buckle portions 118, 128 ensure that the first and second engaging portions 114, 124 are securely engaged with each other, so as to prevent the stethoscope 200 from falling out of the protective case 100 during the shipment.

As shown in FIG. 3, the bump 123 includes a third cavity portion 1231. The third cavity portion 1231 has a third opening 1231a. The second opening 122a and the third opening 1231a are respectively located at opposite sides of the second housing 120.

Reference is made to FIG. 4. FIG. 4 is a cross-section view of the protective case 100 taken along line 4-4 in FIG. 2, in which the stethoscope 200 with a single head is accommodated in the protective case 100.

As shown in FIG. 4, the stethoscope 200 is accommodated in the protective case 100. The stethoscope 200 includes the chest piece 210 and the tube 220. The tube 220 is connected to the chest piece 210. The chest piece 210 is accommodated in the first accommodating space 140. The tube 220 is accommodated in the second accommodating space 150 formed by the first and second notches 111, 121 and extends out of the protective case 100. In the embodiments as illustrated by FIG. 4, the chest piece 210 includes a single head. The bump 123 abuts against a surface 210a of the chest piece 210 opposite to the head. In this way, the bump 123 can help to position the chest piece 210 in the protective case 100 to prevent the chest piece 210 from colliding with the protective case 100 during the shipment and thereby prevent from causing damage to the chest piece 210.

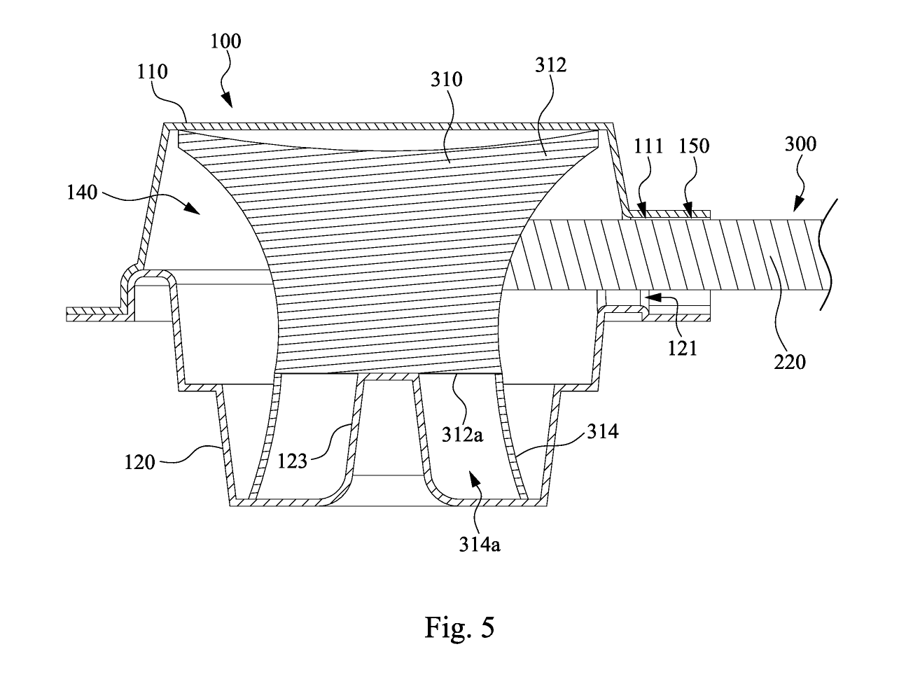

Reference is made to FIG. 5. FIG. 5 is a cross-section view of the protective case 100 taken along line 4-4 in FIG. 2, in which a stethoscope 300 with a double head is accommodated in the protective case 100.

As shown in FIG. 5, the stethoscope 300 is accommodated in the protective case 100. The stethoscope 300 includes a chest piece 310 and a tube 220. Compared with the embodiments as illustrated by FIG. 4, the chest piece 310 of the stethoscope 300 includes double heads. Specifically, the chest piece 310 includes a first head 312 and a second head 314. In present embodiment, the first head 312 is a larger side of the chest piece 310 with a circular edge and a diaphragm thereon. The larger diaphragm side is normally used for adult patients. In present embodiment, the second head 314 is a smaller side of the chest piece 310 with a circular edge, a bell and an acoustic sound hole thereof. The small bell side is normally used for pediatric or thin patients. The second head 314 includes a hollow portion 314a and is connected to a surface 312a of the chest piece 310. The second head 314 tapers toward the first head 312. The bump 123 is abutted against the surface 312a of the chest piece 310 and is accommodated in the hollow portion 314a of the second head 314 while the stethoscope 300 is accommodated in the first accommodating space 140 of the protective case 100. In this way, the bump 123 can help to position the chest piece 310 in the protective case 100 to prevent the chest piece 310 from colliding with the protective case 100 during the shipment and thereby prevent from causing damage to the chest piece 310.

As shown in FIGS. 4 and 5, the protective case 100 of the present disclosure is applicable to stethoscopes with a variety of chest pieces.

According to the foregoing recitations of the embodiments of the disclosure, it can be seen that the protective case of the present disclosure is a package structure light and easy to carry. Therefore, using the protective piece of the present disclosure to pack the stethoscope is convenient for the user to take the stethoscope to everywhere without crashing the chest piece of the stethoscope. The bump located in the protective case helps to position the diverse chest piece so as to prevent the chest piece from colliding with the protective piece and thereby causing damage to the chest piece.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A protective case for a stethoscope, comprising:
a first housing comprising:
a first cavity portion having a first opening; and
a first engaging portion connected to the first opening; and
a second housing comprising:
a second cavity portion having a second opening;
a second engaging portion connected to the second opening and detachably engaged with the first engaging portion such that the first cavity portion and the second cavity portion form an accommodating space therebetween; and
a bump connected to the second cavity portion and protruding toward the second opening.

2. The protective case of claim 1, wherein one of the first engaging portion and the second engaging portion has a protrusion, another of the first engaging portion and the second engaging portion has a slot configured to be detachably engaged with the protrusion.

3. The protective case of claim 1, wherein the first housing further comprises a first connecting portion connected to the first engaging portion, the second housing further comprises a second connecting portion connected to the second engaging portion and the first connecting portion, and the first connecting portion and the second connecting portion form a junction therebetween.

4. The protective case of claim 1, wherein the bump comprises a third cavity portion having a third opening, and the second opening and the third opening are respectively located at opposite sides of the second housing.

5. The protective case of claim 1, wherein the first housing further comprises a notch located at an edge of the first opening, and the notch extends from an inner wall of the first cavity portion to an outer edge of the first engaging portion.

6. The protective case of claim 1, wherein the second housing further comprises a notch located at an edge of the second opening, and the notch extends from an inner wall of the second cavity portion to an outer edge of the second engaging portion.

7. The protective case of claim 1, wherein each of the first cavity portion and the second cavity portion has an annular edge.

8. The protective case of claim 1, wherein the first housing further includes a first buckle portion located at an edge of the first engaging portion, the second housing further includes a second buckle portion located at an edge of the second engaging portion, and the first buckle portion is detachably buckled with the second buckle portion.
